(19) United States
(12) Patent Application Publication
(10) Pub. No.: US 2021/0407091 A1
(43) Pub. Date: Dec. 30, 2021

(54) SYSTEM AND METHOD FOR PRODUCING A TISSUE PATCH FOR USE IN RECONSTRUCTION OF TUBULAR ANATOMICAL STRUCTURES

(71) Applicant: Congenita Limited, Newmarket Suffolk (GB)

(72) Inventors: Nidhin Laji, Newmarket Suffolk (GB); Alessandro Faraci, Newmarket Suffolk (GB); Pablo Lamata De La Orden, Newmarket Suffolk (GB)

(21) Appl. No.: 17/464,037

(22) Filed: Sep. 1, 2021

Related U.S. Application Data
(63) Continuation of application No. PCT/IB2020/051962, filed on Mar. 6, 2020.

(30) Foreign Application Priority Data
Mar. 8, 2019 (GB) 1903154.1

(51) Int. Cl. G06T 7/10; G06T 17/20; B33Y 80/00; B33Y 50/00

(52) U.S. Cl. CPC G06T 7/10; G06T 17/20; B33Y 80/00; G06T 2207/30204; G06T 2207/30172; G06T 2207/10028; G06T 2207/30048; B33Y 50/00

(57) ABSTRACT

Aspects of the invention provide a method of constructing a patch for use in reconstruction of tubular anatomical structures, the method comprising: a) providing by a system including a processor and a graphical user interface acquiring a digital image of a tubular structure; b) displaying the digital image on the graphical user interface; c) segmenting by the system the digital image; d) generating by the system a three dimensional rendered model of the tubular structure based on the segmented digital image and displaying the three dimensional model on the graphical user interface; e) defining by the system an axial central line through the tubular structure; f) identifying by the system one or more incision points on a surface of the model; g) identifying by the system the diameter of the tubular structure, taken from the central line, at each of a plurality of cross sections through the tubular structure; h) simulating by the system one or more cuts through the tubular structure corresponding with the identified incision points; i) determining by the system joining points in each cross section for attachment of a tissue patch thereto; j) determining by the system a required diameter of the tubular structure at each cross section; k) determining by the system the a required diameter of the tissue patch by subtracting the diameter of the tubular structure from the required diameter of the tubular structure; l) generating by the system a model of the tissue patch; m) applying by the system the model of the tissue patch to the model of the tubular structure such that the modelled tissue patch attaches to the model of the tubular structure at each of the joining points.

### CROSS REFERENCE TO RELATED APPLICATIONS

Continuation of International Application No. PCT/162020/051962 filed on Mar. 6, 2020. Priority is claimed from British Application No. 1903154.1 filed on Mar. 8, 2019. Both the foregoing applications are incorporated herein by reference in their entirety.

### STATEMENT REGARDING FEDERALLY SPONSORED RESEARCH OR DEVELOPMENT

Not Applicable

### NAMES OF THE PARTIES TO A JOINT RESEARCH AGREEMENT

Not Applicable

### FIELD

The present invention relates to a system and method for producing a tissue patch for use in reconstruction of tubular anatomical structures.

### BACKGROUND

Birth defects, commonly referred to as congenital disorders, occur in around 3% of newborn babies in developed countries. In other countries this rate can be much higher. Congenital defects result in a significant number of deaths each year, predominantly among young children who may have limited life expectancy depending on the nature of the congenital defect. Congenital defects can include organ anomalies, physical deformation, intellectual disability and developmental disability.

Hypoplastic left heart syndrome is a birth defect that affects normal blood flow through the heart due to one or more structures on the left side of the heart not developing properly during pregnancy. The condition is present at birth but may be diagnosed during pregnancy through routine ultrasound scans. For example, the left ventricle may be undeveloped and/or too small, the mitral valves are not formed and/or are very small, the aortic valve is not formed and/or is very small or the ascending portion of the aorta is underdeveloped and/or is too small.

Hypoplastic left heart syndrome is believed to occur in 0.016% to 0.036% of births with 70% of cases occurring in males and has been reported to account for 4% to 9% of all congenital heart disease. Furthermore, coarctation of the aorta is reported in up to 80% of babies suffering from hypoplastic left heart syndrome. Hypoplastic left heart syndrome is a critical congenital birth defect that is thought to be responsible for up to 25% of deaths within the first week of life.

In a normal heart, the right side of the heart pumps oxygen-poor blood from the heart to the lungs. As a person breathes in, the blood in the lungs is oxygenated and returned to the heart. The left side of the heart then pumps oxygen rich blood to the rest of the body. When a baby is suffering from hypoplastic left heart syndrome, the left side of the heart cannot pump oxygen rich blood to the rest of its body properly.

Hypoplastic left heart syndrome is often not immediately fatal due to the presence of two passages that effectively bypass the left side of the heart at birth. The foramen ovale allows blood to flow from the left atrium to the right side of the heart and the patent ductus arteriosus allows blood to flow from the pulmonary artery to the descending aorta. This is illustrated in FIG.1. Essentially, the right ventricle functions as a combined pulmonary and systemic pump.

In order for this to be viable, all blood must return unobstructed to the right atrium through the atrial septum. After passing through the pulmonary trunk, blood flow from the right ventricle divides into blood flowing to the lungs via the branched pulmonary arteries and blood flowing to the systemic circulation via the patent ductus arteriosus. This is illustrated in FIG. 2. As a result, systemic outflow is inversely proportional to the pulmonary outflow—thus the equilibrium of blood flow through each route is dependent on the ratio of pulmonary to systemic resistance. The circulation of hypoplastic left heart syndrome is sustainable in utero, and it is only after birth that problems arise.

This is due to a natural decline in the pulmonary resistance that takes place in the first few weeks of life. As a result, pulmonary blood flow increases causing a resultant decline in systemic output. Moreover, the systemic outflow is responsible for coronary perfusion, meaning that decreased coronary perfusion and decreased cardiac output also result from this effect. After birth, the condition presents as pulmonary over circulation, systemic hypoperfusion and circulatory collapse.

Alternatively, if pulmonary resistance does not decline, the reduced pulmonary blood flow can result in low arterial oxygenation and metabolic acidosis. Other variations in the condition after birth can depend on the state of the foramen ovale and the ductus arteriosus. Closure or obstruction of the foramen ovale, before or after birth, leads to pulmonary venous obstruction causing a decreased antegrade flow through the lungs, severe hypoxemia and metabolic acidosis—this is uniformly fatal if untreated.

Furthermore, closure or restriction of the ductus arteriosus results in decreased systemic perfusion, acidosis and pulmonary over circulation. Restricted flow across the atria occurs in 20% of patients and restriction of the ductus arteriosus is said to occur in 5% of patients.

These scenarios can be treated by cardiac catheterisation specifically balloon atrial septostomy or stent implantation in the ductus arteriosus. However, even if the atrial septum and ductus arteriosus are unobstructed, the decline in pulmonary resistance causes a need for intervention shortly following birth, as the condition is not sustainable for longer than a few weeks. Indeed, if no surgical intervention is carried out, one month mortality is close to 95%.

There are two main treatment methods for hypoplastic left heart syndrome: i) cardiac transplantation; and ii) staged palliation. Cardiac transplantation can restore normal physiology and haemodynamics but a lack of donor organs makes this treatment method unfeasible in many cases. Staged palliation involves three separate palliative operations: i) the Norwood Procedure, ii) forming the superior cavopulmonary anastomosis; and iii) the Fontan operation.

Staged palliation aims to establish the right ventricle as a combined pulmonary and systemic pump. As well as requiring an atrial septum communication, this approach crucially hinges on low pulmonary resistance enabling blood to pass through the pulmonary circulation without needing extra force from the heart. Deoxygenated blood from the body is directed straight to the lungs by attaching the inferior and superior vena cava to the right pulmonary artery. Essentially, circulation is transformed from parallel to series as illustrated in FIG.3 and FIG. 4. Moreover, by allowing unobstructed systemic flow via the right ventricle coronary circulation is maintained.

### The Norwood Procedure

The Norwood procedure was pioneered in the 1980′s and is the first of the three palliative operations referred to above. A neoaorta is reconstructed from the native aorta, pulmonary trunk and donor pulmonary artery homograft tissue to provide a new systemic outlet whilst maintaining coronary perfusion. An atrial septectomy is also performed to maintain unrestricted flow of oxygenated blood from the pulmonary circulation to the right side of the heart. Creating the correct three dimensional orientations that incorporates all of these elements is extremely difficult even for the most skilled of surgeons. Indeed, the Aristotle and Risk Adjustment for Congenital Heart Surgery (RACHS), a scoring system designed to risk adjust outcomes of congenital cardiac disease surgery, scores the Norwood Procedure at 14.5 points on scale of 1.5 to 15! This scale represents the technical difficulty and risk of morbidity and mortality of a cardiac procedure with the higher the score representing the higher the risk of morbidity and mortality.

The pulmonary trunk is transected, preventing flow from the right heart to the branched pulmonary arteries. In the original Norwood procedure, blood was delivered to the pulmonary circulation via an aortopulmonary shunt called a Blalock-Taussig shunt. This is a Gore-tex® tube that is typically attached to the innominate and pulmonary arteries. Another option for delivering pulmonary circulation is to use a right ventricle to pulmonary artery shunt, known as a Sano shunt. This kind of shunt was pioneered to negate diastolic runoff in order to improve coronary perfusion and reduce cardiac failure, reducing interstage mortality. However, there are concerns over the small ventriculotomy required by Sano shunts, specifically with the risk of causing ventricular arrhythmias. At present, the choice of shunt is based mainly on the surgeon's individual preference.

For most centres, the survival rates 30 days after the Norwood procedure is over 70%. Data from the Paediatric Network Single Ventricle Reconstruction Trial showed that risk factors for death within 30 days of the procedure include: low birth weight, genetic comorbidities, extracorporeal membrane oxygenation and deep hypothermic circulatory arrest for longer periods of time. Risk factors for renal failure, sepsis and increased duration of ventilation included: other genetic abnormalities, lower centre case volumes and an open sternum.

### Forming the Superior Cavopulmonary Anastomosis

The second of the palliative operations involves removal of the shunt implanted during the Norwood procedure and forming of the superior cavopulmonary anastomosis thereafter. The superior cavopulmonary anastomosis can take one of two forms: a bidirectional Glenn shunt or a hemi Fontan. This allows venous drainage from the upper body to directly enter the pulmonary circulation. This procedure is usually performed when the patient is between 4 to 6 months old.

### The Fontan Operation

The third of the palliative operations is known as the Fontan operation. In this procedure, venous drainage from the lower body is channelled to the lungs via an inferior cavopulmonary anastomosis. This is usually carried out when the patient is between 18 and 36 months old. The complete Fontan circulation partially unloads the right ventricle, and reduces stress associated with preparing the unloaded volume of blood through the pulmonary circulation. In order for this circulation to succeed, low pulmonary resistance and unobstructed branched pulmonary arteries are required. Cyanosis is mostly or completely resolved once the Fontan circulation has been established.

Aspects of the present invention thus seek to achieve an optimal arch reconstruction that has the following three main characteristics: i) an aortic diameter wide enough to allow a good conduit function; ii) smooth arch angles and gradual changes of diameter to prevent flow obstacles an inefficiencies; and iii) sufficient inter-aortic distance for the pulmonary arteries to grow.

### SUMMARY

As used in this document, the term native aorta refers to a patient's natural aorta tissue. The term neoaorta refers to the patient's restructured aorta that is a combination of native tissue and a tissue patch stitched thereto.

An aspect of the invention provides a method of constructing a patch for use in reconstruction of tubular anatomical structures, the method comprising:

a) providing, by a system including a processor and a graphical user interface, a digital image of a tubular structure;

b) displaying the digital image on the graphical user interface;

c) segmenting, by the system, the digital image;

d) generating, by the system, a three dimensional rendered model of the tubular structure based on the segmented digital image and displaying the three dimensional model on the graphical user interface;

e) defining, by the system, an axial central line through the tubular structure;

f) identifying, by the system, one or more incision points on a surface of the model;

g) identifying, by the system, the diameter of the tubular structure, taken from the central line, at each of a plurality of cross sections through the tubular structure;

h) simulating, by the system, one or more cuts through the tubular structure corresponding with the identified incision points;

i) determining, by the system, joining points in each cross section for attachment of a tissue patch thereto;

j) determining, by the system, a required diameter of the tubular structure at each cross section;

k) determining, by the system, the required diameter of the tissue patch by subtracting the diameter of the tubular structure from the required diameter of the tubular structure;

l) generating, by the system, a model of the tissue patch; and

m) applying, by the system, the model of the tissue patch to the model of the tubular structure such that the modelled tissue patch attaches to the model of the tubular structure at each of the joining points.

The present invention provides an easy and reproducible method and system for determining dimensions of tissue patches used in reconstructive surgery in patients suffering from congenital defects. The ability to correctly to determine the necessary tissue dimensions of a tissue patch prior to surgery offers potentially positive effects on long term patient health and reduction of mortality as a consequence of surgery. Automating the process of generating tissue patch dimensions has the added benefit of reducing the instances of human error in determining tissue patch dimensions. The tissue patch generated through use of the present invention can be taken into theatre and compared against the intraoperative anatomy of patients for verification prior to reconstruction.

The method may further comprise the step of determining, by the system, a point on the central line corresponding with each incision point identified on the surface of the model, wherein the point on the central line is determined by calculating the distance of all points on the central line from the incision point and selecting the point on the central line with the shortest distance from a respective incision point, and wherein each cross section of step g) is associated with a respective selected point on the central line.

The step of acquiring a digital image of a tubular structure may comprise acquiring an image through use of MRI or CT imaging apparatus.

The step of segmenting, by the system, the digital image may comprise identifying one or more structures from the digital image and applying an identifying marker, or label, to each identified structure.

The step of defining, by the system, the axial centre line through the tubular structure may comprise identifying a plurality of voxels at the centre of the tubular structure and labelling the voxels sequentially from one end of the tubular structure to the other and defining a vector comprising distance and direction to each voxel.

The step of identifying, by the system, one or more incision points on the surface of the model may comprise applying a mesh to the surface of the model and identifying the one or more incision points on the mesh.

The step of identifying, by the system, one or more incision points on the surface of the model may comprise at least one longitudinal incision and at least one resection or transection.

The step of identifying, by the system, the at least one longitudinal incision point on the surface of the model may comprise generating a plurality of cross sections through the tubular structure and identifying a first incision point on the surface of the tubular structure in a first cross section, identifying the point on the central line corresponding with the first incision and identifying a point on the central line corresponding with a second cross section, wherein the first incision point, the point on the central line corresponding with the first cross section and the point on the central line corresponding with the second cross section are used to determine a second incision point corresponding with the point on the central line corresponding with the second cross section.

The method may further comprise the step of joining, by the system, each identified point and displaying a cut line constructed from the at least the first incision point and second point on the graphical user interface.

The step of determining, by the system, joining points in each cross section may comprise manipulating the joining points in three dimensional space until the distance between two joining points in a single cross section is equal to the diameter of the tubular structure.

The method may further comprise generating the tissue patch through additive manufacturing techniques.

The tubular structure may be a vascular structure. The vascular structure may be an aorta.

These and other features of the present invention will be presented in more detail in the following detailed description of the invention and the associated figures.

### BRIEF DESCRIPTION OF THE DRAWINGS

The invention will now be described by way of reference to the following figures:

FIG. 1 illustrates a hypoplastic left heart in which 10 indicates coarctation, 15 indicates ductus arteriosus, 20 indicates branched pulmonary arteries, 25 indicates pulmonary trunk, 30 indicates the left atrium, 35 indicates hypoplastic left ventricle, 40 indicates right ventricle, 45 indicates right atrium, 50 indicates atrial septal defect, 55 indicates diminutive ascending aorta.

FIG. 2 illustrates competing pulmonary and coronary blood flow in a patient with hypoplastic left heart syndrome in which the grey arrow indicates flow to coronaries and the black arrows indicate pulmonary flow as determined by pulmonary resistance.

FIG. 3 illustrates circulation in hypoplastic left heart syndrome (HLHS) where 60=Patent Ductus Arteriosus and 65=Atrial Septal Defect, 70 indicates pulmonary and 75 indicates systemic.

FIG. 4 shows a Fontan circulation diagram where 65=Atrial Septal Defect. 70 indicates pulmonary and 75 indicates systemic.

FIG. 5 illustrates a high level workflow according to an embodiment of the invention in which 80 indicates MRI, 82 indicates segmentation, 84 indicates incision identification, 86 automated design of ideal neoaorta with graft and 88 indicates 3D print of separate patch and native tissue pieces.

FIG. 6 illustrates an exemplary method according to embodiments of the invention in which S1 etc indicate steps of the method.

FIG. 7 illustrates a surgical technique used at the Evelina Children's Hospital to reconstruct a native aorta. A shows incisions, B shows resected and C shows reconstructed.

FIG. 8 Illustrates application of a surgical technique known as Damus-Kaye-Stansel Anastomosis. 1 indicates open aorta, 2 indicates homograft and 3 indicates pulmonary artery trunk.

FIG. 9 shows screenshots of a digital image acquired through MRI both with and without segmentation masks applied.

FIG. 10 shows an example user interface of a system according to embodiments of the invention and a rendered three dimensional model of several tubular anatomical structures.

FIG. 11 shows a rendered three dimensional model of a tubular anatomical structure with several incision points identified on the surface of the model.

FIG. 12 illustrates a process of smoothing the central line of a tubular anatomical structure in which 90 indicates neighbours, 92 indicates average neighbouring coordinates, 94 indicates vectors, 96 indicates selected point and 98 indicated smoothed.

FIG. 13 illustrates reference points used for identification of an incision point on a specific cross section of a tubular anatomical structure. 100 indicates uncut cross section and 102 indicates cut cross section.

FIG. 14 shows an incision line along a tubular anatomical structure that is determined from multiple incision points relating to respective cross sections therethrough.

FIGS. 15A, 15B and 15C show a simulation of the process of opening a native aorta. An incision point on the native aorta is projected onto the three dimensional model of the neoaorta. The neoaorta comprises tissue from the native aorta and a tissue patch.

FIG. 16 shows dimensions of the reconstructed aorta showing a graph indicating thickness of the neoaorta from the proximal to the distal end.

FIG. 17 shows a reconstructed aorta (neoaorta) as a result of a surgical technique that combines the native aorta and pulmonary trunk into a single tube through a double barrel root. 1 indicates open aorta, 2 indicates homograft and 3 indicates pulmonary artery trunk.

FIG. 18 shows a cross section through the double barrel root of FIG. 17.

FIG. 19 illustrates known and unknown variables used to determine the parameters of a tissue patch.

FIGS. 20 and 21 illustrate examples of neoaortas reconstructed using embodiments of the invention.

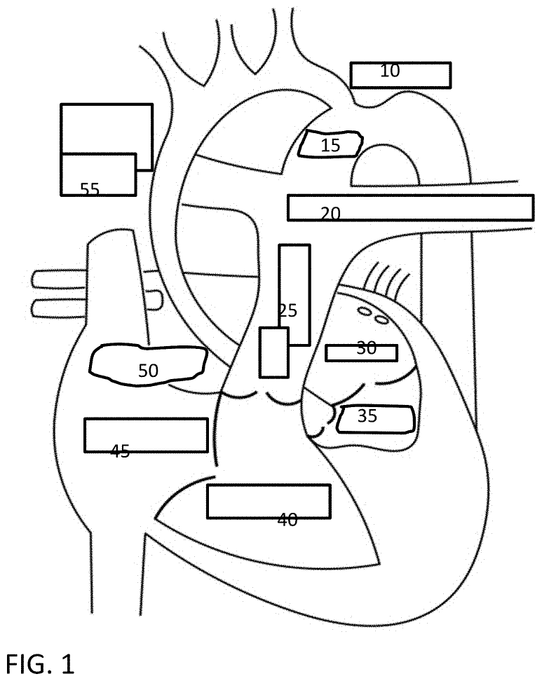

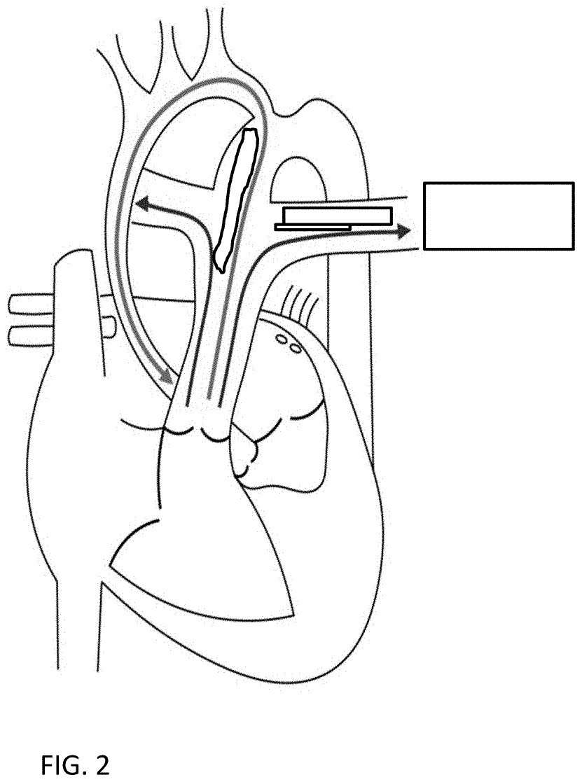

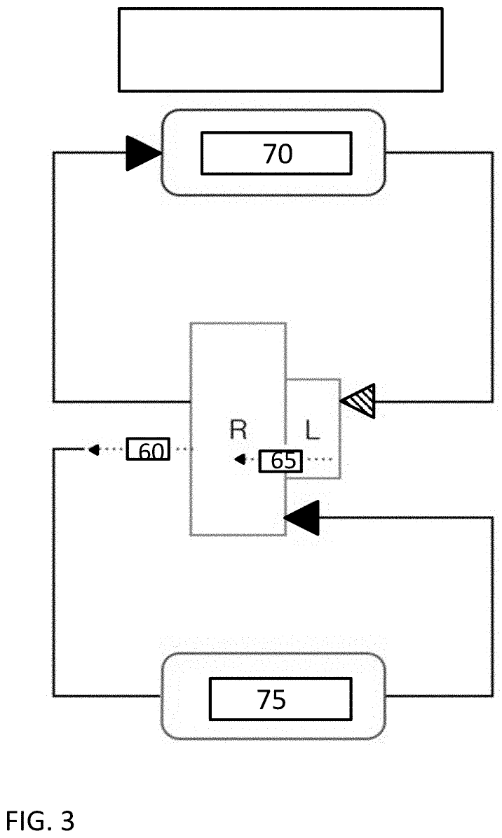

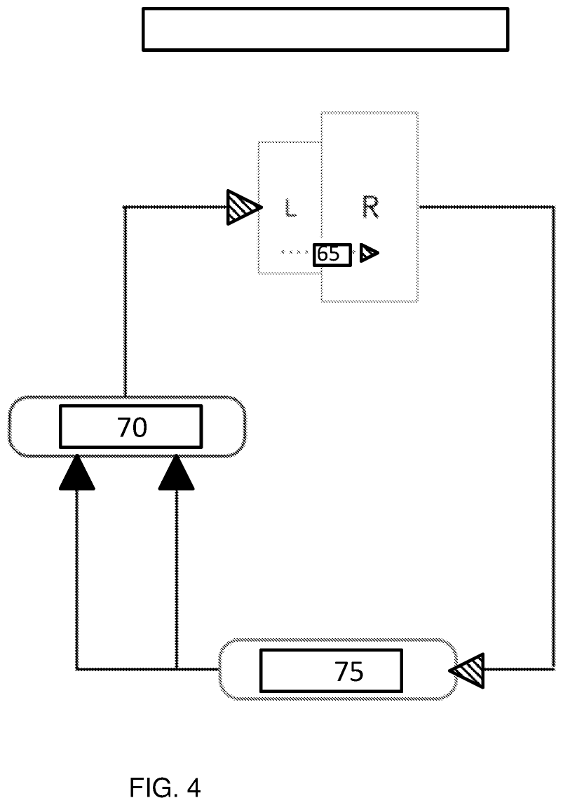

### DETAILED DESCRIPTION

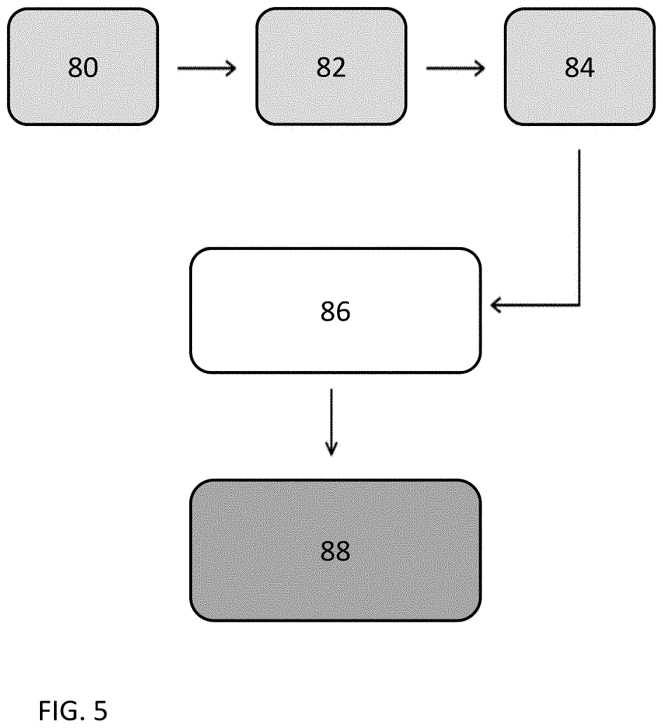

FIG. 5 shows a general workflow for a system according to embodiments of the invention. In general terms the workflow requires the following steps: i) acquisition of a MRI image; ii) segmentation of the MRI image; iii) identifying incision points; iv) automated design of a tissue patch; and v) printing the tissue patch and native tissue pieces using additive manufacturing techniques.

The system comprises a graphical user interface arrangement, a storage arrangement and a processing arrangement. The graphical user interface includes a single graphical user interface or, alternatively, a plurality of graphical user interfaces that may form an integral part of a consumer electronic device such as a smart phone, tablet or computer system. The storage arrangement may include one or more servers or a stand alone storage device. The processing arrangement processes, receives and executes instructions in response to user actions performed on the graphical user interface and in connection with data stored in the storage arrangement.

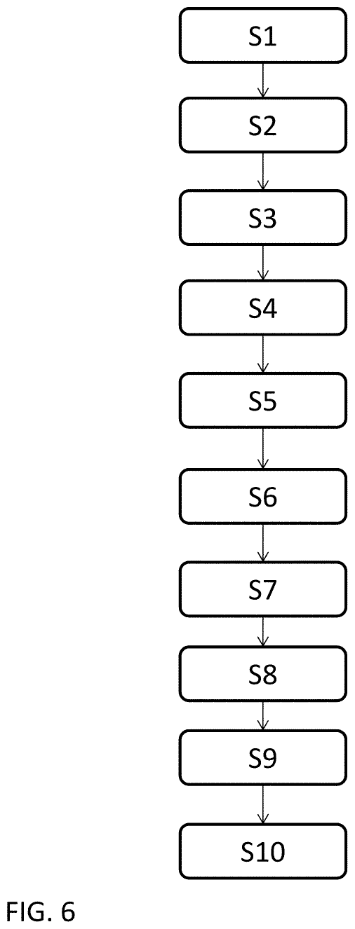

Referring now to FIG. 6 there is illustrated a first embodiment of the method of the invention. According to this embodiment, the method provides that imaging data of a native aorta is captured using appropriate image capture techniques and entered into a surgical mapping system for subsequent processing and modelling of a tissue patch suitable for repairing a patient's native aorta. The surgical mapping system is installed on individual computing devices, i.e. laptops, desktop computer or tablet devices. While description relating to embodiments of the invention refers to reconstruction of a patient's aorta, it will be appreciated that the embodiments of the invention are applicable to reconstruction of other tubular structures within the human body.

The method steps set out below and their order is given as an exemplary example only. Accordingly, some or all of the method steps may be performed and in any order without departing from the scope of the invention.

In a first step S1, a patient is scanned using an appropriate imaging technique to obtain an image of the patient's native aorta. Alternatively, the image may be pre-determined and provided to the method. In this description, MRI acquisition is used to obtain the necessary image or images of the patient's aorta but other alternative imaging techniques, i.e. CT scanning, may be used. The primary purpose of the image is to enable a surgeon to determine the diameter of the aorta to a high degree of accuracy and plot a centre line between two incision points. These geometric features are unique to an individual patient and form the basis of calculations to model a tissue patch suitable for reconstructing the aorta. From here-on-in, a reconstructed aorta incorporating a tissue patch shall be referred to as a neo-aorta.

The image data can be transferred to the surgical mapping system in a number of different ways. In one embodiment, the image data is uploaded to one or more servers operated by a medical facility or group. The image data may be sent directly to the surgical mapping system by way of email, direct transfer or electronic communication protocols such as Near Field Communication or Bluetooth ®. The image data may be stored on a removable storage device and manually transferred to the surgical mapping system. In each case, the image is readable by the surgical mapping system.

MRI works by creating parallel images of a target region of a patient's anatomy from a plurality of coils. Prior to image acquisition, coil calibration is required to generate a coil sensitivity map. The coil sensitivity map quantifies the relative weighting of signals from different points of origin within the reception area of each coil. The data from each coil is processed to derive a raw image. Due to the different sensitivity readings of each coil, the raw image requires further processing to take into account a phenomenon known as aliasing which causes the raw image to distort or warp. The processing steps may be carried out at the time of obtaining the MRI SENSE data in on one embodiment. In another embodiment, the MRI SENSE data may be transferred to the surgical mapping system and processed by a computing device on which the surgical mapping system is installed. In another embodiment, the raw image may be transferred to the surgical mapping system and further processed by a computing device on which the surgical mapping system is installed.

In a second step S2, having been provided with the image, the fully processed image is partitioned into multiple segments in order to modify the fully processed image such that it is easier to analyse. This process is known as segmentation and, in terms of embodiments of the invention, requires that different areas of the image are coloured, textured or assigned an intensity value in order to distinguish target features of the image. For the purposes of embodiments of the invention, the pulmonary artery trunk, stent representing the position of the ductus arteriosus, branched pulmonary arteries, head and neck vessels and coronary arteries are identified through applying respective masks to each structure. Each mask may be a different colour, texture or intensity value, for example.

In a third step S3, a three-dimensional model of the fully processed and segmented image is developed. The three-dimensional model displays one or more of the patient's native aorta, pulmonary artery trunk, stent representing the position of the ductus arteriosus, branched pulmonary arteries, head and neck vessels and coronary arteries. The masks applied in step S2 may be followed through to the three dimensional model.

In a fourth step S4, a central line for the neoaorta is determined. Embodiments of the invention require that the neoaorta follows the same central line as the native arch of the patient's native aorta. The central line is determined by measuring the radius of the native arch of the native aorta at each of a plurality of modelled cross sections. The centre of a circumference of a circle defined by the radius of each modelled cross section is joined in the model to define the central line.

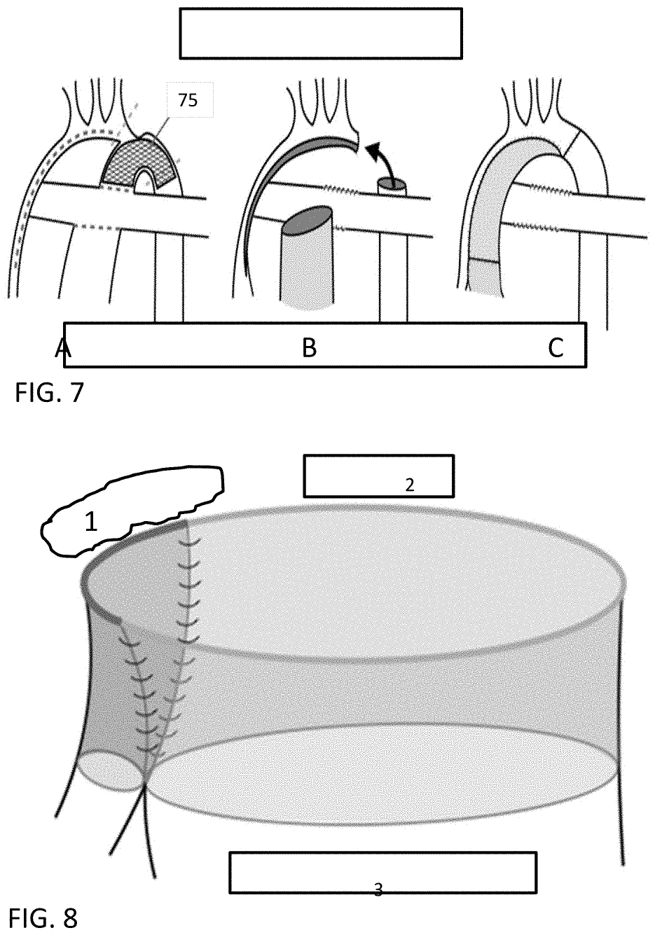

In a fifth step S5, the circumference of the distal and proximal ends of the neo-aorta are determined. The circumference of the distal and proximal ends of the neo-aorta depends on the surgical procedure used. For the purposes of embodiments of the invention, the surgical procedures used at Evalina Children's Hospital to reconstruct a patient's aorta shall be described for illustration only. Such a surgical procedure requires the patient's pulmonary artery and native aorta to be anatomised in a side-by-side fashion as shown in FIG. 7. This results in the pulmonary artery and native aorta forming a double barrel shape at the root of the neo-aorta as shown in FIG. 8. This double barrel transitions to a single tube over a distance of approximately 3-6mm.

In a sixth step S6, a number of virtual incisions are applied to the three-dimensional model. The location and number of incisions is dependent on the surgical technique used. For example, patients suffering from hypoplastic left heart syndrome at Evaline Children's Hospital, the pulmonary trunk is resected and the coarctation of the aorta and ductal tissue are excised. A longitudinal incision is made along the native aorta from the aortic root to the descending thoracic aorta. This incision is substantially straight. A further, triangle shaped, incision is made in the distal end of the ascending aorta and extends 1-1.5cm therein.

In a seventh step S7, the circumference of the native aorta is determined at a plurality of points along its length. The determined circumference is subtracted from a desired circumference to determine the circumference of tissue patch required at each point.

In an eighth step S8, a tissue patch is modelled to extend between the aortic root and the descending thoracic aorta. The size of the tissue patch is determined based on the resected circumference of the aortic root and pulmonary trunk at one end thereof and on the resected circumference of the descending thoracic aorta at the other end thereof. The tissue patch follows the central line determined in step S4. The length of the tissue patch is thus determined by the modelled distance between the combined aortic root and pulmonary trunk and the resected descending thoracic aorta. The circumference of the tissue patch is fixed at each end by the circumferences determined in step S5. The circumference of the tissue patch between each end point is fixed at each of the points referred to in step S7. Any variation in desired circumference between points is accommodated by flexibility in the tissue of the native aorta. A linear transition is provided between adjacent slices. In some embodiments, a linear transition is simply applied between the circumference of the combined aortic root and pulmonary trunk and descending thoracic aorta.

In a ninth step S9, the modelled tissue patch is fitted to the three-dimensional model.

In a tenth step S10, optionally, the modelled tissue patch is produced using additive manufacturing techniques, i.e. 3D printing.

### EXAMPLES

Initial images, pre-Norwood procedure, were obtained at the Evalina Children's Hospital by a SENSE acquisition on a Philips 1.5-Tesla Achieva Scanner. A first pass 3D angiography technique following intravenous injection of an extra-vascular contrast agent was used. Patients were given 0.1 mmol/kg body weight of either gadopentatate dimeglumine or gaderate meglumine. An acceleration factor of 2 was employed with a flip angle of 40° and a breath hold time of 20-30 seconds. A minimum of two phases was acquired. Images had a 200-320 mm field of view and 0.1-1.7 mm isotropic voxel size. Additional flow and CINE data was also acquired. Data was obtained retrospectively and anonymised.

In order to objectively monitor and assess the accuracy of segmentations at later stages of development, a protocol to dictate how images should be segmented was developed. Segmentations were produced using ITK-SNAP version 3.2.0-rc2. Several structures were required as input data for the surgical planner. These included the diminutive ascending aorta, pulmonary artery trunk and descending aorta. Other structures were segmented to aid the process of defining the location of incisions; these included the coronary arteries, branched pulmonary arteries, the stent present in the ductus arteriosus and descending aorta. All of these structures were identified and subsequently segmented individually with separate labels.

The segmentation protocol was designed for ITK-SNAP 3.2.0-rc2. With the exception of the coronary arteries and stent, which are segmented manually, the other vessels are segmented using both manual and semi-automatic techniques.

Levelset segmentation with manual initialization and refinement is the semi-automatic technique used. Thresholding of greyscale values was used to eliminate noise and to make blood vessels more prominent; a lower boundary of 1,000 was adopted. Segmentation initialisation was achieved by placing several bubble cursors of varying radii in the lumen of each vessel. The dynamic growth of the levelset was governed by the weighted contribution between two forces: one maximising the similarity of the intensity within the segmented domain, another minimising the curvature of the edge of the domain. The weights of 1.00 and 0.25 were adopted for each force respectively. The evolution was then run until the vessel could be identified.

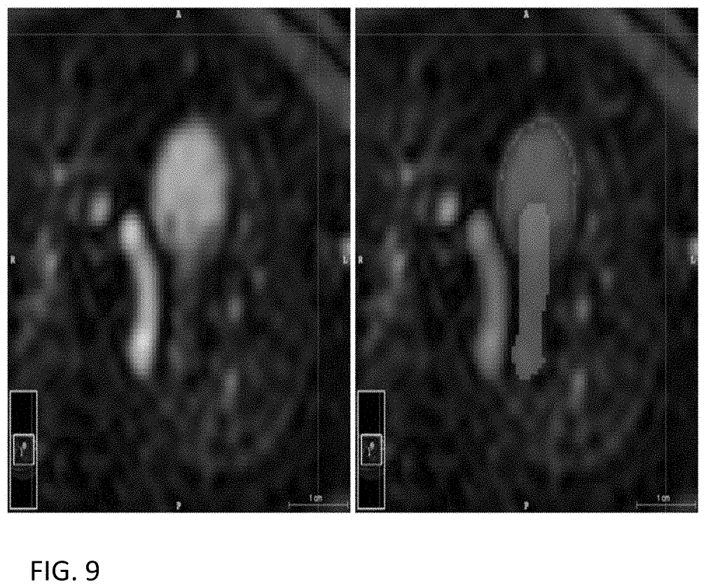

The coronary arteries were segmented manually by painting a mask on each axial slice using a brush size of a single pixel. The vessels were typically visible for four or five axial slices. The stent was also segmented manually by painting the mask on each axial slice. This was achieved using a round shaped brush of varying size. The thickness of the stent varied between patients. FIG. 9 shows an example of an image both with and without segmentation masks applied.

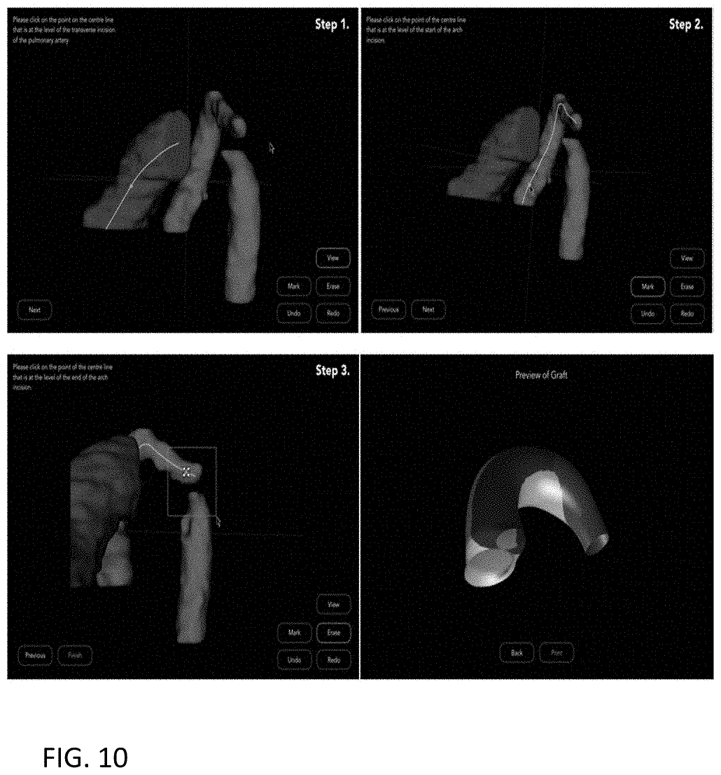

FIG. 10 shows a three-dimensional rendering of a segmentation produced on ITK-SNAP with all necessary structures segmented in different masks.

Segmentations and scans had voxels dimensions 0.6509 mm×0.6509 mm×0.6509 mm. Triangular surface meshes were generated by an isosurface of the segmentation for three-dimensional rendering and interaction purposes. The opacity of the vessels was selected as 0.3 (in a range from 0 to 1), to allow clear visualisation of vessels from any angle: this representation allowed a surgeon to select vertices on the segmentation using the Data Cursor feature in MATLAB_R2014b. Each selected point corresponded to the level of incisions carried out in the arch reconstruction. The coordinate of four incision points were recorded: first, the start of the longitudinal incision along the aorta; second, the level at which the arch is transected proximal to the ductus arteriosus; third, the level at which the descending aorta is transected; and finally, the level at which the pulmonary artery is transected.

Once the incision point data was identified, a central skeleton line through the vessel was computed. This was calculated using the function Skeleton3D, an optimised parallel homotopic thinning algorithm, on MATLAB. The output of this function was a set of voxels that corresponded to the centre of the vessel, ordered by slice. The voxels were labelled sequentially from one end of the vessel to the other. This was achieved by assigning vectors to each voxel that represented the direction and distance of that voxel to the next voxel along the central line. After manually specifying an initial start point at one end of the central line, an algorithm was used to sequentially determine the vector of each voxel on the central line. The algorithm selected the vector with the smallest magnitude between a particular voxel and every other voxel making up the central line without an assigned vector.

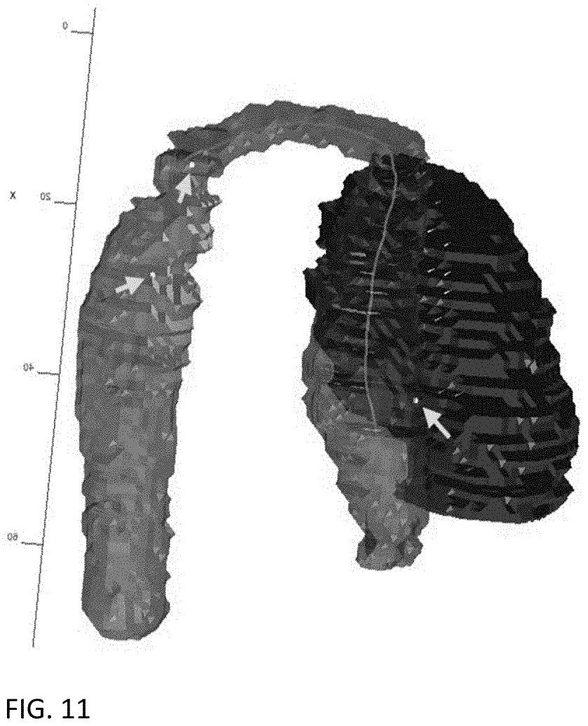

With a central line in place, the next stage of the algorithm is to assign a point on the line corresponding to each incision point indicated on the surface of the mesh. This is the basis of linking the selected incision point to the cross section used to calculate the tissue patch's dimensions. A function calculates the distance between each incision point and every other point on the central line. The voxel on the central line that has the shortest distance to the incision point is then chosen as the corresponding central line voxel (and slice) for the incision point. Example incision points on the surface of a segmented image can be seen in FIG. 11.

Cross sections were defined by selecting neo-aorta triangular surface elements located in the perpendicular plane to the central line vector. The mean distance of the surface point to the central line is an estimation of the radius of each cross section. Data of the radius of each cross section at every point of the central line is finally smoothed using a moving average filter.

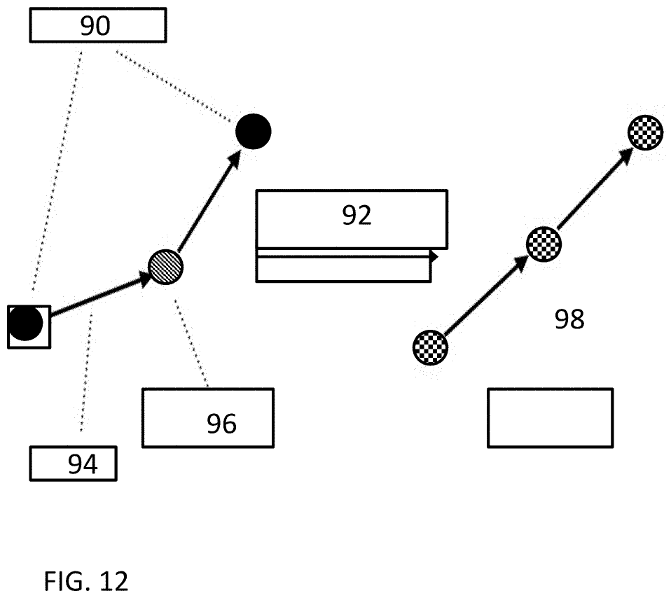

To ensure that the cross sections were completely aligned, the direction of the vectors were also smoothed by a moving average with a window of two neighbours (each point of the central line is moved to a new position that is determined by calculating the average of coordinates of its neighbours either side of it. This smoothing process may be repeated many times. An example of the smoothing process is shown in FIG. 12.

Once the vessels were smoothed and radii values of the cross sections calculated, a simulated incision is carried out. The incision point on each cross section of the aorta is vital for modelling the joining point, or anastomosis, between the tissue patch and the native aorta.

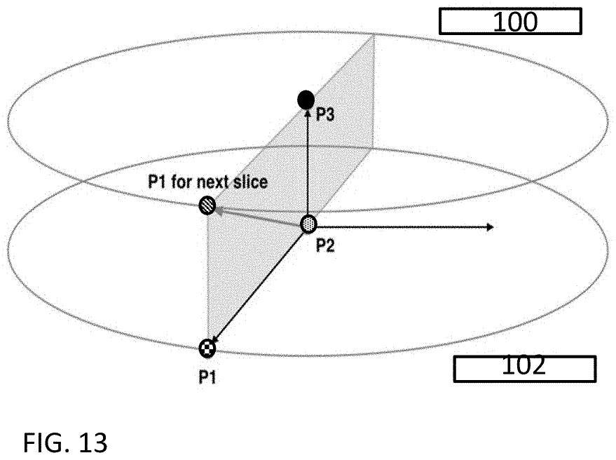

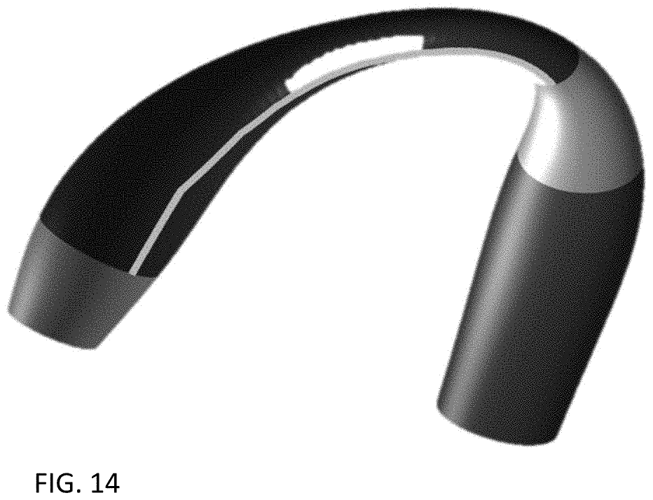

An incision point algorithm was designed and applied to every cross section following the start of the incision. Each cross section is labelled as “cut” or “uncut”, starting with all labelled as “uncut” except for the slice with the defined incision point that is labelled as cut. The incision point of a given uncut cross section is determined from three key points as shown in FIG. 13. The known incision point of the previous cut cross section (P1), the centre of the previous cross section (P2) and the centre of the next uncut cross section. Essentially, the incision point being determined will lie in the plane intersecting P1, P2 and P3 as shown in FIG. 13. Essentially, this plane is the same plane in which the scalpel cuts the native aorta and is thus referred to as the incision plane. The closest point to P1 is chosen from the two points that result from the intersection. Incision points in each cross section are finally joined to form the overall incision along the native aorta as shown in FIG. 14.

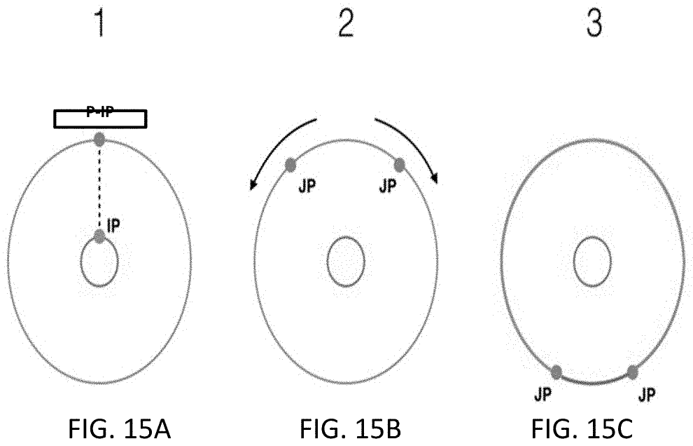

The incision point at each cross section of the native aorta is the basis of calculating the coordinates of the joining points between the tissue patch and the native aorta tissue in the neoaorta. First, a neoaorta of a desired thickness is modelled around the native aorta's central line. Then, as shown in FIG. 15A the incision points from the native aorta model are projected onto the neoaorta. Each cross section's incision point is split into two joining points, as shown in FIG. 15. B that are each moved an equal distance around the neoaorta's circular cross section. This is continued until the section of circumference between the two joining points on the side opposite to the initial incision is equal to the circumference of the native aorta, as shown in FIG. 15C. Lines are drawn between the joining points of a cross section to the joining points of adjacent cross sections to represent the anastomosis between the graft and the native aorta. The isosurface is then re-rendered for the tissue patch and native aorta by constructing the mesh around the two anastomoses.

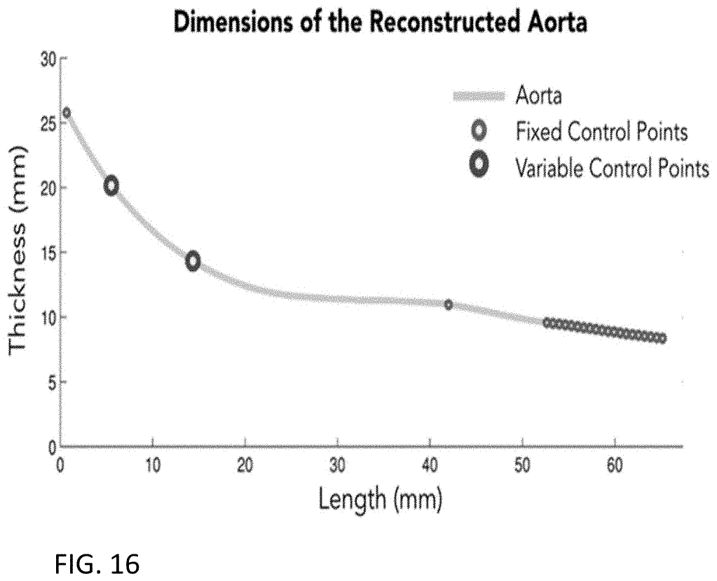

The desired circumference values of cross sections determine the transition in thickness and thus smoothness of the reconstruction. In order to record and manipulate this transition, these desired values were plotted against the slice's length along the central line. The resultant graph, as shown in FIG. 16, was an effective tool to visualise the smoothness of the reconstructed aortic arch. Moreover, by introducing control points, the graph became the interface for determining the curvature and thickness of the reconstructed aortic arch. The graph allowed finer adjustments of the tissue patch shape by allowing specific sections of the reconstruction to be thinned or widened. This process was iterated by visual inspection in collaboration with a surgeon.

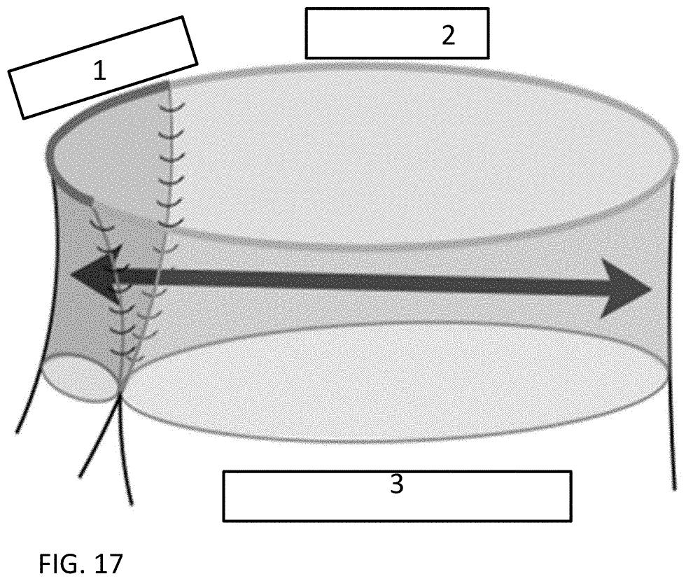

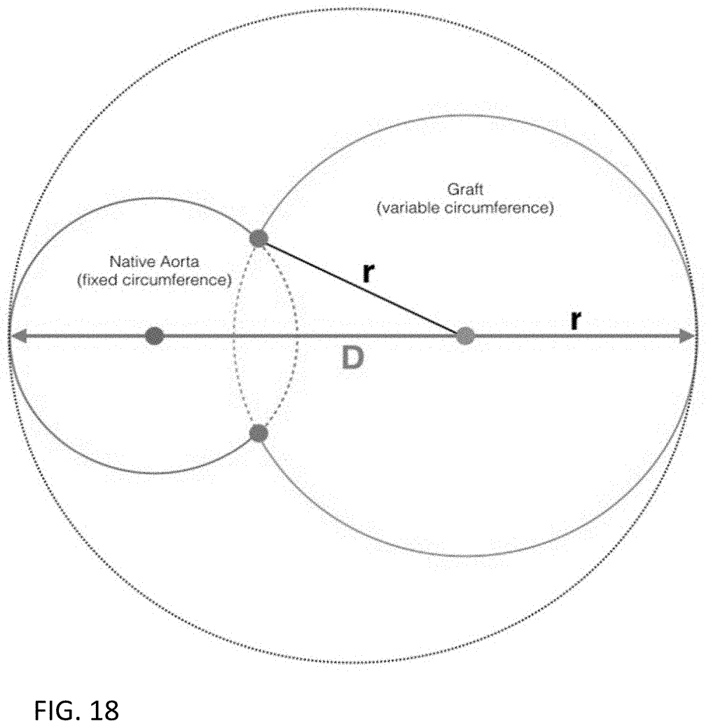

For surgical procedures followed at Evalina Children's Hospital, consideration was required for transition of the double barrelled aortic root to fully circular neoaorta. Such a transition occurs over a distance of approximately 3-6 mm. To take account of this, an additional central line originating from the pulmonary artery was modelled and joined by the central line of the native aorta. To prevent bulging at the base of the neoaorta, desired thickness values were specified to determine the amount of tissue patch in each slice, instead of desired circumference values from aortic and pulmonary roots used for the cylindrical section of the neoaorta. Therefore, the rate at which the central lines of native aorta and neoaorta come together is defined by this thickness, i.e. diameter, of the root of the reconstruction. This is illustrated in FIG. 17 and FIG. 18. Thickness values for each slice were calculated by extrapolating between predetermined values for the most proximal and distal end of the transition. At the most proximal end, this was determined by the combined diameter of the pulmonary artery and the native aorta. At the most distal end this was determined by the diameter of the first completely circular slice of the neoaorta. The transition in diameter, i.e. extrapolation between these two ends of the double barrelled root was set to a linear relationship.

After diameter values for each slice were calculated, the way in which the native aorta opens up following the longitudinal incision was modelled. The algorithm used to model this process used the same function that was applied to native aorta distal to the transition, as described above. The extent to which the native aorta was opened over these transition slices was specified. Although determined arbitrarily, the native aorta was known to require opening fully after 6mm along the central line.

Next, joining points between the tissue patch and the native aorta were identified. In each cross section the known variables are: the coordinates of the joining points, the radius of the opened native aorta and the total desired diameter of the cross section. The unknown variables are the radius of the tissue patch and the distance between the central points of the each circle.

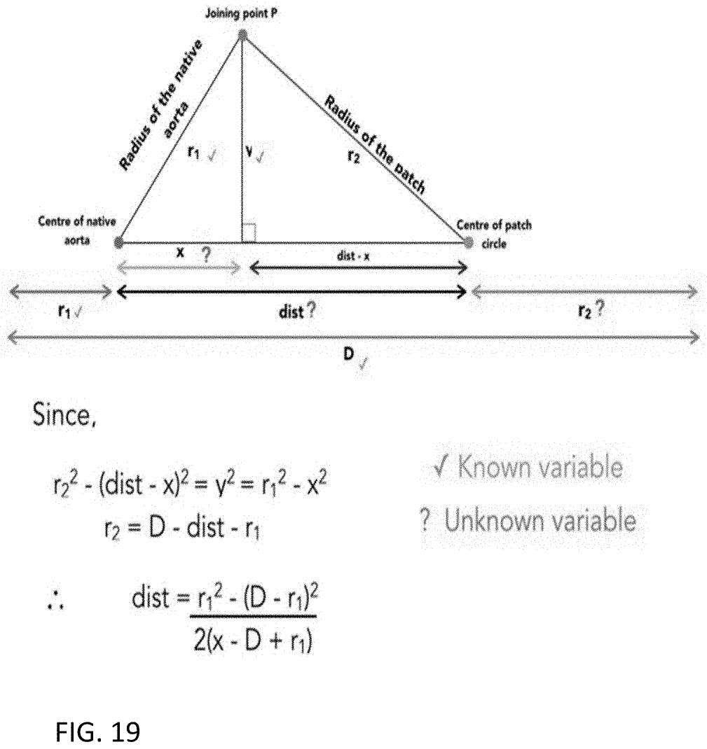

FIG. 19 shows how the distance between the central lines is calculated. The value for y is found by subtracting the y value of the coordinate for the centre of the native aorta from the y value of point p. Using Pythagoras' theorem the value for x can then be determined using values for r₁ and y. Furthermore, y can be expressed in terms of r₂ and (dist-x). In turn, r₂ can also be expressed in terms of D, dist and r₁ and substituted into the expression for y. The resultant formula determines the distance between the two central lines for the desired values of D. This distance allows the software to determine the location of the central point of the circular tissue patch slice, which is used to model the amount of tissue patch material in the slice accordingly.

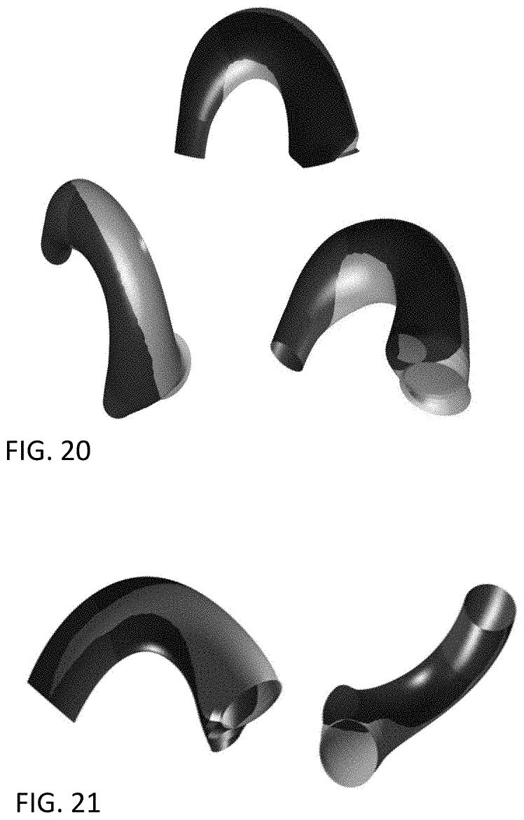

Finally, the anastomosis between the reconstructed aortic arch and descending aorta was represented. This first involved excising a triangle 1-1.5 cm deep into the ascending aorta. The triangular end of the tissue patch was then anastomosed to the sides of the excised area. In order to model the anastomosis, a central line was first calculated for the descending aorta. This was achieved by smoothing the existing central line from the coarctation. Smoothing ensured that the coarctation was no longer present. With the central line in place, the descending aorta was modelled. Each cross section of the anastomosis composed of native aorta and tissue patch graft. Since the shape of the anastomosis was triangular, the amount of tissue patch in each slice was gradually reduced until it was composed entirely of descending aorta thus resulting in a triangular distal patch. FIG. 20. and FIG. 21. demonstrate examples of a reconstructed aorta made up from tissue from the native aorta, tissue patch and tissue from the descending aorta.

The method is an ex vivo method. For example, an in silico method.

In one embodiment there is provided a tissue patch manufactured to the dimensions obtained by the method disclosed herein.

While exemplary embodiments have been set forth above for the purposes of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages may exist even though they may not have been explicitly discussed herein.

In the context of this specification “comprising” is to be interpreted as “including”.

Approximately as employed herein means±10%.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

1. A ex vivo method of constructing a tissue patch for use in reconstruction of tubular anatomical structures, the method comprising:
a. providing, by a system including a processor and a graphical user interface, a digital image of a tubular structure;
b. displaying the digital image on the graphical user interface;
c. segmenting, by the system, the digital image;
d. generating, by the system, a three dimensional rendered model of the tubular structure based on the segmented digital image and displaying the three dimensional model on the graphical user interface;
e. defining, by the system, an axial central line through the tubular structure;
f. identifying, by the system, one or more incision points on a surface of the model;
g. identifying, by the system, the diameter of the tubular structure, taken from the central line, at each of a plurality of cross sections through the tubular structure;
h. simulating, by the system, one or more cuts through the tubular structure corresponding with the identified incision points;
i. determining, by the system, joining points in each cross section for attachment of a tissue patch thereto;
j. determining, by the system, a required diameter of the tubular structure at each cross section;
k. determining, by the system, a required diameter of the tissue patch by subtracting the diameter of the tubular structure from the required diameter of the tubular structure;
l. generating, by the system, a model of the tissue patch; and
m. applying, by the system, the model of the tissue patch to the model of the tubular structure such that the modelled tissue patch attaches to the model of the tubular structure at each of the joining points.

2. The method according to claim 1, further comprising the step of determining, by the system, a point on the central line corresponding with each incision point identified on the surface of the model, wherein the point on the central line is determined by the system by calculating the distance of all points on the central line from the incision point and selecting the point on the central line with the shortest distance from a respective incision point, and wherein each cross section of step g) is associated with the selected point on the central line.

3. The method according to claim 1, wherein the step of acquiring, by the system, a digital image of a tubular structure comprises acquiring an image through use of imaging apparatus.

4. The method according claim 1, wherein the step of segmenting, by the system, the digital image comprises identifying one or more structures from the digital image and applying an identifying marker, or label, to each identified structure.

5. The method according to claim 1, wherein the step of defining, by the system, the axial centre line through the tubular structure comprises identifying a plurality of voxels at the centre of the tubular structure and labelling the voxels sequentially from one end of the tubular structure to the other and defining a vector comprising distance and direction to each voxel.

6. The method according to claim 1, wherein the step of identifying, by the system, one or more incision points on the surface of the model comprises applying a mesh to the surface of the model and identifying the one or more incision points on the mesh.

7. The method according to claim 1, wherein the step of identifying, by the system, one or more incision points on the surface of the model comprises identifying incision points suitable for at least one longitudinal incision and at least one resection or transection.

8. The method according to claim 7, wherein the step of identifying, by the system, the at least one longitudinal incision point on the surface of the model comprises generating a plurality of cross sections through the tubular structure and identifying a first incision point on the surface of the tubular structure in a first cross section, identifying the point on the central line corresponding with the first incision and identifying a point on the central line corresponding with a second cross section, wherein the first incision point, the point on the central line corresponding with the first cross section and the point on the central line corresponding with the second cross section are used to determine a second incision point corresponding with the point on the central line corresponding with the second cross section.

9. The method according to claim 8, wherein the method further comprises the step of joining each identified point and displaying a cut line constructed from the at least the first incision point and second point on the graphical user interface.

10. The method according to claim 1, wherein the step of determining, by the system, joining points in each cross section comprises manipulating the joining points in three dimensional space until the distance between two joining points in a single cross section is equal to the diameter of the tubular structure.

11. The method according to claim 1, wherein the method further comprises generating, by the system, the tissue patch through additive manufacturing techniques.

12. The method according to claim 1, wherein the tubular structure is a vascular structure.

13. The method according to claim 12, wherein the vascular structure is an aorta.

14. The method according to claim 13, further comprising the steps of:
i) modelling, by the system, a first additional central line originating from a first adjacent tubular structure;
ii) joining, by the system, the first additional central line of the first adjacent tubular structure to a first end of the axial central line of the tubular structure;
iii) defining, by the system, a transition between the tubular structure and first adjacent tubular structure;
iv) determining, by the system, a parameter value at a first end of the transition ;
v) determining, by the system, a parameter value at a second end of the transition;
vi) applying, by the system, a linear transition between the parameter value of the first end of the transition and the parameter value of the second end of the transition; and
vii) determining, by the system, a radius of the modelled patch at each end of the transition.

15. The method according to claim 14, wherein the adjacent tubular structure is a pulmonary artery and the combined aorta and pulmonary artery define a diameter D1, and wherein the diameter D2 of the combined tubular structure and tissue patch is equal to Dl.

16. The method according to claim 15, further comprising the steps of:
viii) modelling, by the system, a second additional central line originating from a second adjacent tubular structure;
ix) excising, by the system, a triangle into the second adjacent tubular structure;
x) identifying, by the system, the diameter of the second adjacent tubular structure, taken from the central line, at each of a plurality of cross sections through the second adjacent tubular structure in the region of the triangular excision;
xi) determining, by the system, joining points in each cross section through the second adjacent tubular structure for attachment of the tissue patch thereto; and
xii) determining, by the system, the required diameter of the tissue patch by subtracting the diameter of the second adjacent tubular structure at each cross section from the required diameter of the tubular structure.

17. The method according to claim 14, wherein the adjacent tubular structure is a descending aorta and the patch defines a triangle shaped interface therewith, wherein a parameter of the patch at one end of the interface has a value of 1 and the corresponding parameter of the patch at the other end of the interface has a value of 0 and wherein the transition between values is substantially linear along the length of the interface.

18. A tissue patch manufactured to dimensions obtained using the method according to claim 1
